(19) United States
(12) Patent Application Publication
(10) Pub. No.: US 2026/0355076 A1
(43) Pub. Date: Nov. 12, 2026

(54) OIL-IN-WATER COSMETIC COMPOSITION COMPRISING SALT AND POLYALKYLSILSESQUIOXANE

(71) Applicant: LG H&H CO., LTD., Seoul (KR)

(72) Inventors: Dong Won SHIN, Seoul (KR); Soo Jung HA, Seoul (KR); Young Bong KIM, Seoul (KR)

(73) Assignee: LG H&H CO., LTD., Seoul (KR)

(21) Appl. No.: 19/487,397

(22) PCT Filed: May 24, 2024
(86) PCT No.: PCT/KR2024/007069

§ 371 Date: Nov. 24, 2025

(30) Foreign Application Priority Data
May 24, 2023 (KR) 10-2023-0067034

(51) Int. Cl. A61K 8/06; A61K 8/20; A61K 8/34; A61K 8/365; A61K 8/41; A61K 8/42; A61K 8/44; A61K 8/49; A61K 8/58; A61K 8/81; A61Q 19/00

(52) U.S. Cl. CPC A61K 8/062; A61K 8/20; A61K 8/345; A61K 8/365; A61K 8/41; A61K 8/42; A61K 8/44; A61K 8/494; A61K 8/585; A61K 8/8147; A61Q 19/00; A61K 2800/805

(57) ABSTRACT

The present invention provides an oil-in-water (O/W) cosmetic composition that comprises a salt in an aqueous phase part, which is a continuous phase, and comprises polyalkylsilsesquioxane in an oil phase part, which is a discontinuous phase, and does not contain a separate emulsifier. As oil phase components have a predetermined particle size and are uniformly and stably dispersed in the aqueous phase part, the oil-in-water (O/W) cosmetic composition of the present invention can maintain its formulation and provide excellent stability over time, a differentiated appearance, and an excellent feeling of use when spread on the skin.

### TECHNICAL FIELD

The present invention relates to an oil-in-water (O/W) cosmetic composition which comprises an oil phase part including polyalkylsilsesquioxane; and an aqueous phase part including a salt.

### BACKGROUND ART

Cosmetic compositions are generally prepared by mixing aqueous and oil components. Since the aqueous and oil components are immiscible, emulsification techniques are applied to ensure that the aqueous and oil components can be mixed well in a single formulation in the preparation of the cosmetic composition. In particular, technologies using emulsifiers are commonly utilized. However, when the type and amount of emulsifier are not appropriate, the emulsifying ability may decrease, thereby leading to separation of the oil and aqueous phases. Also, depending on the emulsifier, a sticky feeling of use may be imparted, or irritation may occur.

To solve the above problems, a technique has been developed in which an oil phase is prepared in the form of oil capsules and uniformly dispersed in an aqueous phase without using emulsifiers. However, this technique has a problem in that a cosmetic composition may cause a foreign-body sensation when applied onto the skin due to cellulose, polysaccharide gum, or carrageenan contained in the membrane of the oil capsule.

To resolve the foreign-body sensation caused by the oil capsules, a technique has been developed in which a cosmetic composition is prepared in the form of oil droplets using waxes or elastomers that readily melt on the skin. However, this technique has a problem in that the formulation provides a heavy feeling of use and the preparation process becomes complicated by including a high-temperature treatment step.

Accordingly, there is a need for a technology for a cosmetic composition capable of stably dispersing an oil phase part in an aqueous phase part without using an emulsifier and also providing a pleasant feeling of use.

### DISCLOSURE

### Technical Problem

The present inventors completed an oil-in-water (O/W) cosmetic composition which does not contain an emulsifier as an effective ingredient, maintains its stable formulation by uniformly dispersing an oil phase part in an aqueous phase part, and provides an excellent feeling of use.

The present invention is directed to providing an oil-in-water (O/W) cosmetic composition which comprises, an oil phase part including polyalkylsilsesquioxane and is in the form of oil droplets with an average particle diameter of 0.01 to 5 mm; and an aqueous phase part including a salt.

### Technical Solution

Hereinafter, a composition according to the present invention will be described in more detail.

An oil-in-water (O/W) cosmetic composition according to the present invention comprises, an oil phase part including polyalkylsilsesquioxane and is in the form of oil droplets with an average particle diameter of 0.01 to 5 mm; and an aqueous phase part including a salt.

As used herein, the term “oil-in-water (O/W)” refers to a type of dispersion in which an oil phase (a discontinuous phase) is dispersed as fine particles in an aqueous phase (a continuous or external phase).

In the present invention, the oil-in-water (O/W) cosmetic composition can maintain the shape of the oil phase part using the electrostatic repulsion caused by a polarity difference between the continuous phase and the discontinuous phase, wherein the oil phase part can be dispersed in the aqueous phase part without coalescing. This principle may be described with reference to FIG. 2.

In the present invention, when the coefficient of friction (N, force) of the oil-in-water (O/W) cosmetic composition is measured using a friction coefficient tester (FPT-F1, Labthink), it is confirmed that the stickiness index and coefficient of friction of the cosmetic composition are improved even without using a fatty alcohol wax or an oil gelling agent.

In the present invention, the state in which the oil phase part is dispersed in the form of particles means that the oil phase part comprised in the cosmetic composition is uniformly dispersed in the form of spherical particles in the aqueous phase part.

As used herein, the term “oil droplet” does not mean only a mathematically perfect spherical shape but may also include a shape whose projection is identical or similar to a circle or an ellipse.

As used herein, the term “dispersion” means that an internal phase is evenly spread throughout an external phase. Particularly, it preferably means that the oil phase part is in the form of oil droplets, has a predetermined size, and is evenly distributed at a predetermined ratio per unit area in the aqueous phase part.

In the present invention, the oil-in-water (O/W) cosmetic composition forms oil droplets having a uniform size. In addition, the cosmetic composition is quite stable in various conditions, including immediately after being prepared and after being stored under various storage situations such as storage at room temperature for 30 days after preparation, storage in a 50 °C constant temperature bath for 30 days after preparation, storage with exposure to a fluorescent light for 2 weeks after preparation, and storage with freezing and thawing repeated 5 times.

In the present invention, the oil-in-water (O/W) cosmetic composition may not comprise an emulsifier, or the emulsifier may not be substantially included based on the total weight of the cosmetic composition, and the “not substantially included” may mean included at less than 1.0 wt %, 0.1% or less, 0.01% or less, or 0.001% or less or not included, but the present invention is not limited thereto.

In the present invention, the polyalkylsilsesquioxane may include one or more selected from the group consisting of polymethylsilsesquioxane, polypropylsilsesquioxane, and polycaprylylsilsesquioxane, but the present invention is not limited thereto.

In the present invention, the salt may be one or more selected from the group consisting of 2-[4-(2-hydroxyethyl)piperazin-1-yl]ethanesulfonic acid (HEPES), urea, citric acid, sodium chloride (NaCl), disodium EDTA, trisodium EDTA, and sodium citrate. Preferably, two or more of the above group may be used, and more preferably, one or more of HEPES, urea, citric acid, and sodium citrate may be used, but the present invention is not limited thereto.

As used herein, the term “salt” refers to a raw material that can be ionized in the aqueous phase part and thus exhibit+/−ionic properties.

In the present invention, the “salt” is included to increase the polarity of the aqueous phase and increase the repulsive force with the oil phase. In addition, the “salt” may be used to adjust the pH, but the present invention is not limited thereto.

In the present invention, polyalkylsilsesquioxane may be included in an amount of 0.01 to 1 wt %, 0.01 to 0.8 wt %, 0.01 to 0.6 wt %, 0.01 to 0.4 wt %, 0.01 to 0.2 wt %, 0.01 to 0.1 wt %, 0.02 to 1 wt %, 0.02 to 0.8 wt %, 0.02 to 0.6 wt %, 0.02 to 0.4 wt %, 0.02 to 0.2 wt %, 0.05 to 1 wt %, 0.05 to 0.8 wt %, 0.05 to 0.6 wt %, 0.05 to 0.4 wt %, 0.05 to 0.2 wt %, 0.05 to 0.1 wt %, 0.08 to 1 wt %, 0.08 to 0.8 wt %, 0.08 to 0.6 wt %, 0.08 to 0.4 wt %, 0.08 to 0.2 wt %, 0.08 to 0.1 wt %, 0.1 to 1 wt %, 0.1 to 0.8 wt %, 0.1 to 0.6 wt %, 0.1 to 0.4 wt %, 0.1 to 0.2 wt %, for example, 0.1 wt % based on the total weight of the cosmetic composition, but the present invention is not limited thereto.

In the present invention, the salt may be included in an amount of 0.1 to 20 wt %, 0.1 to 15 wt %, 0.1 to 10 wt %, 0.1 to 5 wt %, 0.1 to 3 wt %, 0.1 to 2 wt %, 0.1 to 1 wt %, 0.1 to 0.5 wt %, for example, 0.1 wt % based on the total weight of the cosmetic composition, but the present invention is not limited thereto.

In the present invention, the aqueous phase and oil phase of the oil-in-water (O/W) cosmetic composition may be transparent or opaque. To highlight the state in which the oil phase is dispersed in the aqueous phase, it is preferable that the aqueous phase is transparent, and in this case, the aqueous phase may have a transmittance of 80 to 99%, 80 to 95%, 85 to 99%, 85 to 95%, 90 to 99%, 90 to 95%, for example, 95% in the visible wavelength range, but the present invention is not limited thereto.

In one experimental example of the present invention, the oil-in-water (O/W) cosmetic composition achieved a stable formulation by using a solution different from that of the related art. Therefore, unlike the related art, the use of a wax or an oil gelling agent was not essential, and as a result, stickiness or a coefficient of friction could be improved by not adding a wax or an oil gelling agent to the cosmetic composition.

In the present invention, the oil-in-water (O/W) cosmetic composition may not comprise one or more selected from the group consisting of a wax and an oil gelling agent, or one selected from the group consisting of a wax and an oil gelling agent may not be substantially included based on the total weight of the cosmetic composition, and the “not substantially included” may mean included at less than 1.0 wt %, 0.1% or less, 0.01% or less, or 0.001% or less or not included, but the present invention is not limited thereto.

In the present invention, since the oil-in-water (O/W) cosmetic composition does not contain or does not substantially include any wax or oil gelling agent, the stickiness or coefficient of friction of the cosmetic composition can be improved, and a stable formulation can be exhibited.

In the present invention, the oil-in-water (O/W) cosmetic composition may further include one or more selected from the group consisting of an oil, a water-soluble thickener, a polyol, an emollient, an acidity adjusting agent, and a solvent, but the present invention is not limited thereto.

In the present invention, in the case of the oil-in-water (O/W) cosmetic composition, oil droplets may be more stably maintained by increasing the viscosity of the aqueous phase.

In the present invention, the oil may be included in the oil phase part and may be one or more selected from the group consisting of an ester-based oil, a hydrocarbon-based oil, a silicone-based oil, and a vegetable oil, but the present invention is not limited thereto.

In the present invention, the ester-based oil may be an ester compound of a fatty acid and a fatty alcohol. Preferably, the ester-based oil may be one or more selected from the group consisting of isopropyl myristate, isopropyl palmitate, cetyl octanoate, cetyl 2-ethylhexanoate, 2-octyl dodecyl myristate, and di-isostearyl malate, but the present invention is not limited thereto.

In the present invention, the hydrocarbon-based oil may be one or more selected from the group consisting of petroleum-based oils such as liquid paraffin, Vaseline, and isoparaffins; synthetic oils such as hydrogenated polydecene, synthetic squalane, and polybutene; and plant-derived oils such as vegetable squalane and hydrogenated squalane, but the present invention is not limited thereto.

In the present invention, the silicone-based oil may be an oil having a siloxane bond (Si—O—Si). Preferably, the silicone-based oil may be one or more selected from the group consisting of dimethicone, dimethiconol, phenyl trimethicone, disiloxane, cyclomethicone, cyclopentasiloxane, and cyclohexasiloxane, but the present invention is not limited thereto.

In the present invention, the vegetable oil may be an oil obtained from plants present in nature. Preferably, the vegetable oil may be one or more selected from the group consisting of meadowfoam seed oil, olive oil, rosehip fruit oil, avocado oil, camellia oil, apple seed oil, grape seed oil, sunflower seed oil, macadamia seed oil, castor oil, jojoba seed oil, and caprylic/capric triglyceride, but the present invention is not limited thereto.

In the present invention, the type of water-soluble thickener is not limited as long as it can increase the viscosity of the aqueous phase. Preferably, the thickener may be one or more selected from the group consisting of an acrylate/C10-30 alkyl acrylate crosspolymer, an ammonium acryloyldimethyltaurate/VP copolymer, sodium polyacrylate, polyacrylate crosspolymer-6, and xanthan gum, but the present invention is not limited thereto.

In the present invention, the type of polyol is not limited as long as it is easily soluble in water. Preferably, the polyol may be one or more selected from the group consisting of polyglycerin-3 and propanediol, but the present invention is not limited thereto.

In the present invention, the type of emollient is not limited as long as it softens the skin, increases flexibility, and improves the feeling of use of a product. Preferably, the emollient may be one or more selected from the group consisting of isononyl isononanoate, isopropyl palmitate, dicaprylyl carbonate, hydrogenated polyisobutene, glyceryl rosinate, and sodium polyacrylate, but the present invention is not limited thereto.

In the present invention, the acidity adjusting agent may be one or more selected from the group consisting of tromethamine and sodium citrate, but the present invention is not limited thereto.

In the present invention, the solvent is a substance for dissolving each component of the cosmetic composition. Preferably, the solvent may be one or more selected from the group consisting of purified water, propanediol, benzyl alcohol, ethanol, propylene glycol, butylene glycol, 1,2-hexanediol, butoxyethanol, butyl acetate, n-butyl alcohol, octamethylcyclotetrasiloxane, ethyl acetate, and oleyl alcohol, but the present invention is not limited thereto.

In the present invention, in addition to the above-described components, the oil-in-water (O/W) cosmetic composition may further include any one or more selected from the group consisting of oil components, moisturizers, polymeric materials, saccharides, antioxidants, buffers, various extracts, preservatives, colorants, and fragrances, which are typically used in cosmetics, within a range that does not hinder the effects of the present invention.

Preferably, all the components described in the present disclosure do not exceed the maximum usage amount prescribed by the relevant laws and regulations of Korea, China, the United States, Europe, Japan, or the like (e.g., the Regulations on Safety Standards for Cosmetics in Korea and the Technical Regulations for Cosmetics Safety in China). In other words, preferably, the composition for enhancing the delivery of an active ingredient according to the present disclosure includes the components according to the present disclosure within the content limits permitted by the relevant laws and regulations of each country.

### Advantageous Effects

The present invention provides an oil-in-water (O/W) cosmetic composition which includes a salt in an aqueous phase part (a continuous phase) and polyalkylsilsesquioxane in an oil phase part (a discontinuous phase) and does not contain a separate emulsifier. As oil phase components have a predetermined particle size and are uniformly and stably dispersed in the aqueous phase part, the cosmetic composition can maintain its formulation and provide excellent stability over time, a differentiated appearance, and an excellent feeling of use when spread on the skin.

### DESCRIPTION OF DRAWINGS

FIG. 1 shows the shape of oil droplets immediately after preparation of cosmetic compositions of Examples 1 to 4 and Comparative Examples 1 to 4 according to one experimental example of the present invention.

FIG. 2 is a schematic diagram of the electrostatic repulsion between an aqueous phase part and an oil phase part according to one experimental example of the present invention.

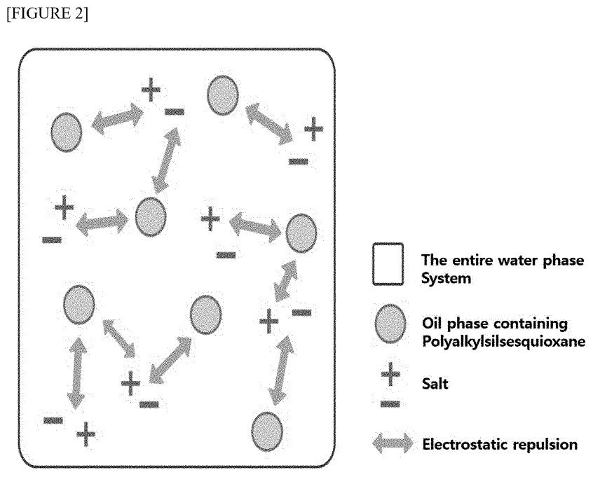

### MODES OF THE INVENTION

Hereinafter, the present invention will be described in detail with reference to examples to help understand the present invention. However, examples of the present invention may be modified into several different forms, and the scope of the present invention is not limited to the examples to be described below. The examples of the present invention are provided to more completely explain the present invention to those with average knowledge in the art. The raw materials used in the present invention were purchased from conventional suppliers and used.

### Preparation Example 1. Preparation of Cosmetic Compositions of Examples and Comparative Examples

### 1-1. Preparation of Cosmetic Compositions of Examples

Cosmetic compositions of examples having the compositions shown in the following Table 1 were prepared. The units are expressed in wt %.

TABLE 1

| No. | Classification | Component name | Example 1 | Example 2 | Example 3 | Example 4 |
|---|---|---|---|---|---|---|
| 1 | Oil phase part | Isononyl isononanoate | 4 | 4 | 4 | 4 |
| 2 | Oil phase part | Meadowfoam seed oil | 2 | 2 | 2 | 2 |
| 3 | Oil phase part | Polyalkylsilsesquioxane | 0.1 | 0.08 | 0.1 | 1 |
| 4 | Aqueous | Carbomer | 0.2 | 0.2 | 0.2 | 0.2 |
| | phase part | | | | | |
| 5 | Aqueous | Tromethamine | 0.16 | 0.16 | 0.16 | 0.16 |
| | phase part | | | | | |
| 6 | Aqueous | Propanediol | 5 | 5 | 5 | 5 |
| | phase part | | | | | |
| 7 | Aqueous | Polyglycerin-3 | 2 | 2 | 2 | 2 |
| | phase part | | | | | |
| 8 | Aqueous | Urea | 0.1 | 0.5 | 0.5 | 0.5 |
| | phase part | | | | | |
| 9 | Aqueous | HEPES | 0.1 | 0.5 | 0.5 | 0.5 |
| | phase part | | | | | |
| 10 | Aqueous | Purified water | To 100 | To 100 | To 100 | To 100 |
| | phase part | | | | | |

Specifically, the raw materials for an aqueous phase part excluding the thickener shown in Table 1 were evenly dispersed in an Agi mixer at a low stirring speed of 1,500 rpm or less for 15 minutes. Next, a thickener was dispersed together with the pre-mixed raw materials for an aqueous phase part at 3,000 rpm for 30 minutes. Finally, the raw materials for an oil phase part were slowly added and completely stirred, and then any foam incorporated into the resulting liquid was removed to prepare cosmetic compositions of Examples 1 to 4.

### 1-2. Preparation of Cosmetic Compositions of Comparative Examples

Cosmetic compositions of comparative examples were prepared in the same manner as in the examples. Each of Comparative Examples 1 to 4 has the composition shown in the following Table 2, and the units are expressed in wt %.

TABLE 2

| | | | Comparative | Comparative | Comparative | Comparative |
|---|---|---|---|---|---|---|
| No. | Classification | Component name | Example 1 | Example 2 | Example 3 | Example 4 |
| 1 | Oil phase part | Isononyl isononanoate | 4 | 4 | 4 | 4 |
| 2 | Oil phase part | Meadowfoam seed oil | 2 | 2 | 2 | 2 |
| 3 | Oil phase part | Polyalkylsilsesquioxane | — | 0.05 | — | 0.1 |
| 4 | Aqueous phase | Carbomer | 0.2 | 0.2 | 0.2 | 0.2 |
| | part | | | | | |
| 5 | Aqueous phase | Tromethamine | 0.16 | 0.16 | 0.16 | 0.16 |
| | part | | | | | |
| 6 | Aqueous phase | Propanediol | 5 | 5 | 5 | 5 |
| | part | | | | | |
| 7 | Aqueous phase | Polyglycerin-3 | 2 | 2 | 2 | 2 |
| | part | | | | | |
| 8 | Aqueous phase | Urea | — | — | 0.5 | — |
| | part | | | | | |
| 9 | Aqueous phase | HEPES | — | — | 0.5 | — |
| | part | | | | | |
| 10 | Aqueous phase | Purified water | To 100 | To 100 | To 100 | To 100 |
| | part | | | | | |

### Experimental Example 1. Evaluation of Formulation Characteristics Immediately after Preparation

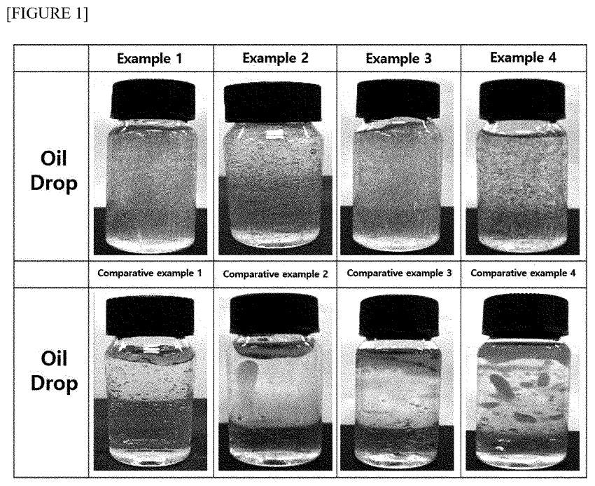

Immediately after preparation of Examples 1 to 4 and Comparative Examples 1 to 4, whether oil droplets were properly formed was visually observed, and the results are shown in FIG. 1.

As can be seen in FIG. 1, in the case of Examples 1 to 4, oil droplets with a uniform size were formed. On the other hand, in the case of Comparative Examples 1 to 3, no oil droplets were formed, and in the case of Comparative Example 4, oil droplets were formed, but were not properly dispersed in the aqueous phase and floated to the top of the composition.

### Experimental Example 2. Evaluation of Formulation Stability

Immediately after preparation of the cosmetic compositions of the examples and comparative examples, and after storage of the cosmetic compositions under storage conditions such as storage at room temperature for 30 days after preparation, storage in a 50 °C constant temperature bath for 30 days after preparation, storage with exposure to a fluorescent light for 2 weeks after preparation, and storage with freezing and thawing repeated 5 times, whether oil droplets were properly formed and maintained was visually observed, and the results are shown in the following Table 3.

TABLE 3

| Stability | Example 1 | Example 2 | Example 3 | Example 4 |
|---|---|---|---|---|
| Immediately after | Stable | Stable | Stable | Stable |
| preparation | | | | |
| Room temperature | Stable | Stable | Stable | Stable |
| 50 °C constant | Stable | Stable | Stable | Stable |
| temperature bath | | | | |
| Exposure to fluorescent | Stable | Stable | Stable | Stable |
| light for 2 weeks | | | | |
| Freezing and thawing | Stable | Stable | Stable | Stable |
| repeated 5 times | | | | |

As can be seen in Table 3, Examples 1 to 4 exhibited uniform stable oil droplets immediately after preparation, and after storage of the cosmetic compositions under storage conditions such as storage at room temperature for 30 days after preparation, storage in a 50 °C constant temperature bath for 30 days after preparation, storage with exposure to a fluorescent light for 2 weeks after preparation, and storage with freezing and thawing repeated 5 times.

### Experimental Example 3. Evaluation of Stickiness Index and Coefficient of Friction

The stickiness index and coefficient of friction of Example 1 and a control were evaluated.

The control used in this evaluation was a cosmetic composition including a fatty alcohol and an oil gelling agent in an oil phase part, that is, a wax-drop cosmetic composition prepared using a wax solidification principle and an oil gelling agent after melting a fatty alcohol through heating and uniformly dispersing the melted fatty alcohol in an aqueous phase. The composition of the control is shown in the following Table 4.

TABLE 4

| No. | Classification | Component name | Control |
|---|---|---|---|
| 1 | Oil phase part | Triethylhexanoin | 1.7 |
| 2 | Oil phase part | Cetearyl alcohol | 0.3 |
| 3 | Oil phase part | Behenyl alcohol | 0.4 |
| 4 | Oil phase part | Polyamide-8 | 0.6 |
| 5 | Aqueous phase part | Carbomer | 0.45 |
| 6 | Aqueous phase part | Tromethamine | 0.16 |
| 7 | Aqueous phase part | Pentylene glycol | 12.5 |
| 8 | Aqueous phase part | Purified water | To 100 |

For comparison between Example 1 and the control, each cosmetic composition was applied onto artificial leather, the cosmetic composition was repeatedly pressed and released using a universal testing machine (TA.XT plus texture analyzer, Stable Micro Systems, UK) to measure the vertical force over time, and the degree of stickiness was evaluated by calculating the area under the curve (AUC), which is known to correlate with stickiness. In addition, after applying the cosmetic composition onto the artificial leather, the coefficient of friction (N, force) of the cosmetic composition was measured using a friction coefficient tester (FPT-F1, Labthink), and the results are shown in the following Table 5.

TABLE 5

| Item | Control | Example 1 |
|---|---|---|
| Stickiness index (AUC) | 2.2 | 0.1 |
| Coefficient of friction (N) | 1.335 | 0.867 |

As can be seen in Table 5, in the case of Example 1, it can be confirmed that the stickiness index and the coefficient of friction were improved, even without using a fatty alcohol wax or an oil gelling agent, compared to the control.

1. An oil-in-water (O/W) cosmetic composition comprising:
an oil phase part including polyalkylsilsesquioxane and is in the form of oil droplets with an average particle diameter of 0.01 to 5 mm; and
an aqueous phase part including a salt.

2. The oil-in-water (O/W) cosmetic composition of claim 1, wherein the oil-in-water (O/W) cosmetic composition does not include an emulsifier, or wherein the oil-in-water (O/W) cosmetic composition includes the emulsifier in an amount of less than 1.0 wt % based on the total weight of the cosmetic composition.

3. The oil-in-water (O/W) cosmetic composition of claim 1, wherein the polyalkylsilsesquioxane further includes one or more selected from the group consisting of polymethylsilsesquioxane, polypropylsilsesquioxane, and polycaprylylsilsesquioxane.

4. The oil-in-water (O/W) cosmetic composition of claim 1, wherein the salt is one or more selected from the group consisting of 2-[4-(2-hydroxyethyl)piperazin-1-yl]ethanesulfonic acid (HEPES), urea, citric acid, sodium chloride (NaCl), disodium EDTA, trisodium EDTA, and sodium citrate.

5. The oil-in-water (O/W) cosmetic composition of claim 1, wherein the polyalkylsilsesquioxane is included in an amount of 0.01 to 1 wt % based on the total weight of the cosmetic composition.

6. The oil-in-water (O/W) cosmetic composition of claim 1, wherein the salt is included in an amount of 0.1 to 20 wt % based on the total weight of the cosmetic composition.

7. The oil-in-water (O/W) cosmetic composition of claim 1, wherein the oil-in-water (O/W) cosmetic composition does not include one or more selected from the group consisting of a wax and an oil gelling agent, or wherein the oil-in-water (O/W) cosmetic composition includes one selected from the group consisting of a wax and an oil gelling agent in an amount of less than 1.0 wt % based on the total weight of the cosmetic composition.
